(19) United States
(12) Patent Application Publication
(10) Pub. No.: US 2018/0153464 A1
(43) Pub. Date: Jun. 7, 2018

(54) ALERTING USER TO ADJUST CLOTHING LAYERS BASED ON SENSED TEMPERATURE AND HUMIDITY

(71) Applicant: International Business Machines Corporation, Armonk, NY (US)

(72) Inventors: Ilse M. Breedvelt-Schouten, Manotick (CA); Jana H. Jenkins, Raleigh, NC (US); Jeffrey A. Kusnitz, Campbell, CA (US); John A. Lyons, Ottawa (CA)

(21) Appl. No.: 15/370,935

(22) Filed: Dec. 6, 2016

(51) Int. Cl. A61B 5/00; A41D 1/00

(52) U.S. Cl. CPC A61B 5/4266; A41D 1/002; A61B 5/742; A61B 5/74; A61B 5/7405; A61B 5/0008

(57) ABSTRACT

A method, system and computer program product for assisting the user to wear an appropriate amount of clothing layers. A smart device (e.g., smartwatch) receives measurements of temperature and humidity sensed at various locations on a human body which are sources of sweat. In response to the measured temperature or humidity being above the first threshold (indication that the user will start to feel overheated) or below the second threshold (indication that the user will start to feel cold), the smart device generates a notification to the user to adjust the clothing layers (e.g., remove a clothing layer). Alternatively, the smart device may receive measurements of a pH level of the user's sweat. The smart device generates a notification to the user to stop exercising and to drink water in response to the received measurement being below a threshold (indication that the user is approaching dehydration).

### TECHNICAL FIELD

The present invention relates generally to physiological monitoring, and more particularly to alerting the user to adjust clothing layers (e.g., add a clothing layer, remove a clothing layer, zip a clothing layer, unzip a clothing layer) based on sensed temperature and humidity using sensors placed within a base clothing layer worn by a user.

### BACKGROUND

People who live in cold climates may often wear multiple layers of clothing, where clothing must at the same time transfer moisture, provide warmth, and protect from wind and rain. Using more or fewer layers, or replacing one layer but not others, allows for flexible clothing to match the needs of each situation. Two thin layers can be warmer yet lighter than one thick layer, because the air trapped between layers serves as thermal insulation.

Dressing in layers is important in maintaining a good core body temperature, especially when engaging in physical activities. Layers allow the people to add or remove pieces of clothing to avoid overheating while continuing to stay warm.

Problems may occur if you overheat in cold temperatures because the effectiveness of the insulation is greatly reduced and you can quickly become chilled. In extreme situations, hyperthermia can result.

For example, a common problem with people unused to physical exertion during the winter is forgetting to remove clothing layer(s) while they are exercising before they start to sweat. By forgetting to remove clothing layer(s), the person may be subject to overheating.

Currently, there is not a means for informing the user as to when to adjust the clothing layers so as to prevent situations, such as overheating.

### SUMMARY

In one embodiment of the present invention, a method for assisting a user to wear an appropriate amount of clothing layers comprises receiving measurements of temperature and humidity sensed at various locations on a human body which are sources of sweat. The method further comprises determining whether the measured temperature or the measured humidity is above a first threshold or is below a second threshold. The method additionally comprises generating, by a processor, a notification to the user on a smart device indicating to adjust clothing layers in response to the measured temperature or the measured humidity being above the first threshold or below the second threshold.

Other forms of the embodiment of the method described above are in a system and in a computer program product.

In another embodiment of the present invention, a method for assisting a user to maintain a balanced pH level comprises receiving measurements of a pH level sensed at various locations on a human body which are sources of sweat. The method further comprises determining whether the measured pH level is below a threshold. The method additionally comprises generating, by a processor, a notification to the user on a smart device indicating to drink water in response to the measured pH level being below the threshold.

Other forms of the embodiment of the method described above are in a system and in a computer program product.

The foregoing has outlined rather generally the features and technical advantages of one or more embodiments of the present invention in order that the detailed description of the present invention that follows may be better understood. Additional features and advantages of the present invention will be described hereinafter which may form the subject of the claims of the present invention.

### BRIEF DESCRIPTION OF THE DRAWINGS

A better understanding of the present invention can be obtained when the following detailed description is considered in conjunction with the following drawings, in which:

FIG. 1 illustrates a physiological monitoring system configured in accordance with an embodiment of the present invention;

FIG. 2 illustrates various layers of clothing worn by a user, including sensors located within a base clothing layer, in accordance with an embodiment of the present invention;

FIG. 3 illustrates a hardware configuration of a smart device which is representative of a hardware environment for practicing the present invention;

FIG. 4 is a flowchart of a method for assisting the user to wear an appropriate amount of clothing in accordance with an embodiment of the present invention; and

FIG. 5 is a flowchart of a method for assisting the user to maintain a balanced pH level in accordance with an embodiment of the present invention.

### DETAILED DESCRIPTION

The present invention comprises a method, system and computer program product for assisting the user to wear an appropriate amount of clothing layers. In one embodiment of the present invention, a smart device (e.g., smartphone, phablet, tablet, smartwatch, smart band, smart glasses, smart key chain, etc.) receives measurements of temperature and humidity sensed at various locations on a human body which are sources of sweat. The smart device determines whether the measured temperature or humidity is above a first threshold (indication that the user will start to feel overheated) or below a second threshold (indication that the user will start to feel too cold). In response to the measured temperature or humidity being above the first threshold or below the second threshold, the smart device generates a notification to the user on the smart device to adjust the clothing layers (e.g., add a clothing layer, remove a clothing layer, zip a clothing layer or unzip a clothing layer). In this manner, the present invention assists the user in wearing an appropriate amount of clothing layers, such as informing the user to remove a clothing layer to prevent overheating or adding a clothing layer to prevent coldness, so that the user is appropriately clothed and comfortable. Alternatively, the smart device may receive measurements of a pH level of the user's sweat. The smart device generates a notification to the user on the smart device to stop exercising and to drink water in response to the received measurement being below a threshold (e.g., pH of 6) (indication that the user is approaching dehydration). In this manner, the present invention assists the user in maintaining a balanced pH level thereby preventing the user from reaching a state of dehydration.

In the following description, numerous specific details are set forth to provide a thorough understanding of the present invention. However, it will be apparent to those skilled in the art that the present invention may be practiced without such specific details. In other instances, well-known circuits have been shown in block diagram form in order not to obscure the present invention in unnecessary detail. For the most part, details considering timing considerations and the like have been omitted inasmuch as such details are not necessary to obtain a complete understanding of the present invention and are within the skills of persons of ordinary skill in the relevant art.

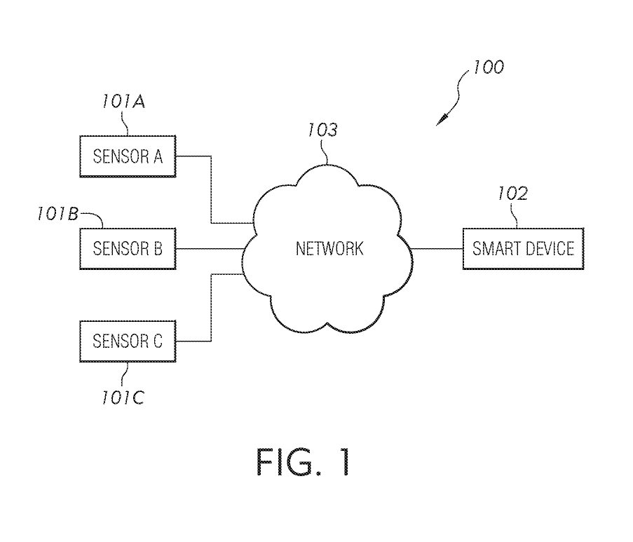

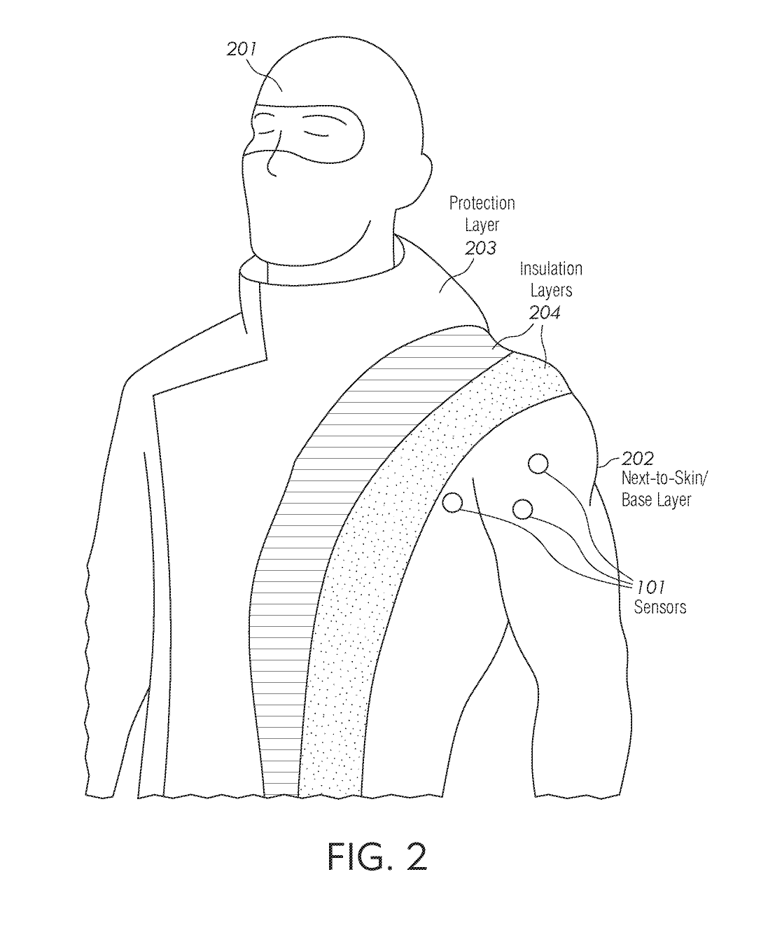

Referring now to the Figures in detail, FIG. 1 illustrates a physiological monitoring system 100 for practicing the principles of the present invention in accordance with an embodiment of the present invention. System 100 includes a plurality of sensors 101A-101C (identified as “Sensor A,” “Sensor B,” and “Sensor C,” respectively in FIG. 1) configured to measure temperature or humidity (such as relative humidity) at various locations on a human body, such as sources of sweat (e.g., armpits, back) as discussed further below in connection with FIG. 2, which depicts the locations of such sensors 101A-101C in a base clothing layer worn by a user. Sensors 101A-101C may collectively or individually be referred to as sensors 101 or sensor 101, respectively. In one embodiment, such sensors 101 are optical fiber Bragg grating based sensors to measure body temperature, such as discussed in Li et al., “Wearable Sensors in Intelligent Clothing for Measuring Body Temperature Based on Optical Fiber Bragg Grating,” Optics Express, Vol. 20, No. 11, 2012, pp. 11740-11752. In another embodiment, such sensors 101 are sensors in the HIH series manufactured by Honeywell® to measure humidity/temperature.

In another embodiment, sensors 101 are configured to measure the pH levels of a user's sweat. In one embodiment, such sensors 101 are ion-sensitive field-effect transistor biosensors. An ion-sensitive field-effect transistor is used for measuring ion concentrations in a solution, such as sweat. When the ion concentration, such as a hydrogen ion changes, the current through the transistor will change accordingly.

Sensors 101 of the present invention are to include any type of biosensor with the capability of measuring temperature, humidity or pH levels and transmitting such information to a smart device as discussed below.

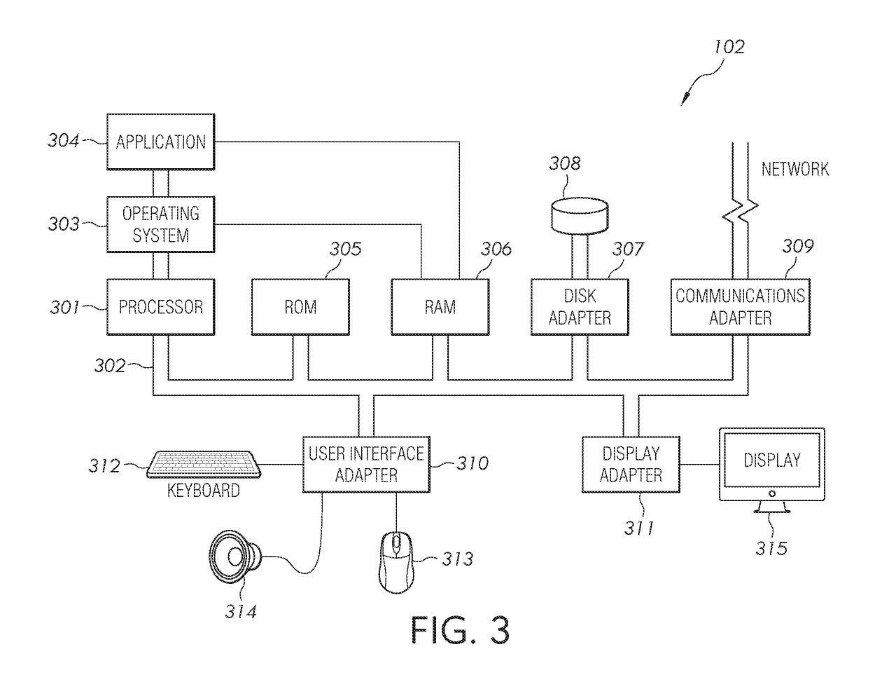

Referring again to FIG. 1, sensors 101 are configured to transmit their measurements to a smart device 102 via a network 103. Smart device 102 may be any type of smart computing device (e.g., smartphone, phablet, tablet, smartwatch, smart band, smart glasses, smart key chain, etc.) configured with the capability of receiving measurements from sensors 101, such as via network 103. A description of the hardware configuration of an exemplary smart device 102 is provided below in connection with FIG. 3.

Network 103 may be, for example, a Fabric Area Network (FAN), a wireless wide area network, a Global System for Mobile Communications (GSM) network, a Wireless Application Protocol (WAP) network, a WiFi network, an IEEE 802.11 standards network, various combinations thereof, etc. Other networks, whose descriptions are omitted here for brevity, may also be used in conjunction with system 100 of FIG. 1 without departing from the scope of the present invention.

System 100 is not to be limited in scope to any one particular network architecture. System 100 may include any number of sensors 101, smart devices 102 and networks 103.

Referring now to FIG. 2, FIG. 2 illustrates various layers of clothing worn by a user 201, including sensors 101 (FIG. 1) located within a base clothing layer 202, in accordance with an embodiment of the present invention. As illustrated in FIG. 2, user 201 may be wearing multiple clothing layers above base clothing layer 202 embedded with sensors 101, such as an outer protection layer 203 and one or more insulation layers 204 between the outer protection layer 203 and base clothing layer 202. Such a user 201 may wear multiple layers of clothing when the outside temperature is cold. To prevent overheating, such as when user 201 is exercising when the outside temperature is cold, the present invention instructs user 201 to adjust the clothing layers to prevent overheating as to well to ensure user 201 stays warm.

In one embodiment, sensors 101 are strategically placed within base clothing layer 202 to measure temperature, humidity or pH levels of the user's sweat at locations where a user typically seats, such as at the armpits and along the back of user 201.

As discussed herein, sensors 101 measure temperature, humidity and/or pH levels of the user's sweat which are transmitted to smart device 102. Smart device 102 determines whether the measured temperature or humidity is above a first designated threshold or below a second designated threshold. In one embodiment, such thresholds may be user-designated. If the measured temperature or humidity is above the first designated threshold (indication that user 201 will start to feel overheated), then smart device 102 informs user 201 via a notification on smart device 102 to remove a clothing layer, such as one of the insulation layers 204, or unzip one of the clothing layers, such as insulation layer 204 as discussed in further detail below. Alternatively, if the measured temperature or humidity is below the second designated threshold (indication that user 201 will start to feel too cold), then smart device 102 informs user 201 via a notification on smart device 102 to add a clothing layer, such as one of the insulation layers 204, or zip up one of the clothing layers, such as insulation layer 204.

In another example, if the measure pH level of the user's sweat is below a designated threshold (e.g., pH level of 6) (indication that user 201 is approaching dehydration), then smart device 102 informs user 201 via a notification on smart device 102 to take a drink of water to prevent dehydration (as well as to stop exercising in case user 201 is exercising). In one embodiment, such a threshold is user-specified.

A description of the hardware configuration of an exemplary smart device 102 is provided below in connection with FIG. 3.

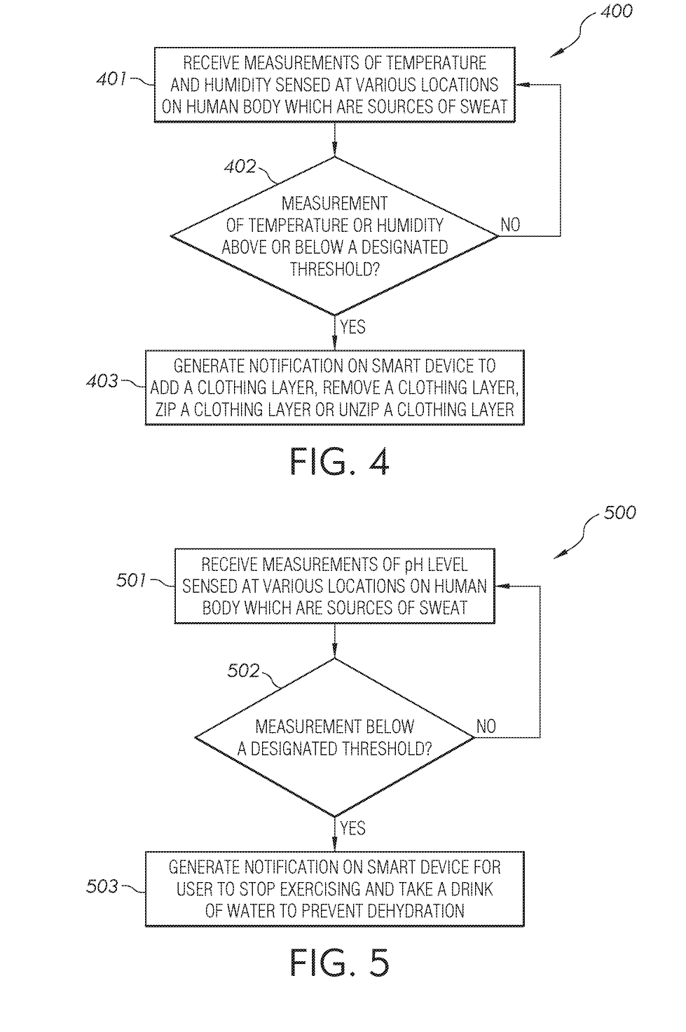

FIG. 3 illustrates a hardware configuration of smart device 102 (FIG. 1) which is representative of a hardware environment for practicing the present invention. Referring to FIG. 3, smart device 102 has a processor 301 coupled to various other components by system bus 302. An operating system 303 runs on processor 301 and provides control and coordinates the functions of the various components of FIG. 3. An application 304 in accordance with the principles of the present invention runs in conjunction with operating system 303 and provides calls to operating system 303 where the calls implement the various functions or services to be performed by application 304. Application 304 may include, for example, a program for assisting user 201 (FIG. 2) to wear an appropriate amount of clothing or a program for maintaining a balanced pH level as discussed further below in connection with FIGS. 4-5.

Referring again to FIG. 3, read-only memory (“ROM”) 305 is coupled to system bus 302 and includes a basic input/output system (“BIOS”) that controls certain basic functions of smart device 102. Random access memory (“RAM”) 306 and disk adapter 307 are also coupled to system bus 302. It should be noted that software components including operating system 303 and application 304 may be loaded into RAM 306, which may be smart device's 102 main memory for execution. Disk adapter 307 may be an integrated drive electronics (“IDE”) adapter that communicates with a disk unit 308, e.g., disk drive. It is noted that the programs for assisting user 201 to wear an appropriate amount of clothing or to maintain a balanced pH level, as discussed further below in connection with FIGS. 4-5, may reside in disk unit 208 or in application 204.

Smart device 102 may further include a communications adapter 309 coupled to bus 302. Communications adapter 309 interconnects bus 302 with an outside network (e.g., network 103 of FIG. 1) thereby enabling smart device 102 to communicate with sensors 101 (FIG. 1).

I/O devices may also be connected to smart device 102 via a user interface adapter 310 and a display adapter 311. Keyboard 312, mouse 313 and speaker 314 may all be interconnected to bus 302 through user interface adapter 310. A display monitor 315 may be connected to system bus 302 by display adapter 311. In this manner, a user is capable of inputting to smart device 102 through keyboard 312 or mouse 313 and receiving output from smart device 102 via display 315 or speaker 314. Other input mechanisms may be used to input data to smart device 102 that are not shown in FIG. 3, such as display 315 having touch-screen capability and keyboard 312 being a virtual keyboard. Smart device 102 of FIG. 3 is not to be limited in scope to the elements depicted in FIG. 3 and may include fewer or additional elements than depicted in FIG. 3.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

As stated in the Background section, dressing in layers is important in maintaining a good core body temperature, especially when engaging in physical activities. Layers allow the people to add or remove pieces of clothing to avoid overheating while continuing to stay warm. Problems may occur if you overheat in cold temperatures because the effectiveness of the insulation is greatly reduced and you can quickly become chilled. In extreme situations, hyperthermia can result. For example, a common problem with people unused to physical exertion during the winter is forgetting to remove clothing layer(s) while they are exercising before they start to sweat. By forgetting to remove clothing layer(s), the person may be subject to overheating. Currently, there is not a means for informing the user as to when to adjust the clothing layers so as to prevent situations, such as overheating.

The principles of the present invention provide a means for assisting the user to wear an appropriate amount of clothing layers, such as informing the user to remove a clothing layer to prevent overheating, as discussed further below in connection with FIG. 4. FIG. 4 is a flowchart of a method for assisting the user to wear an appropriate amount of clothing. Furthermore, as discussed herein, the principles of the present invention provide a means for assisting the user to maintain a balanced pH level so as to prevent dehydration as discussed further below in connection with FIG. 5. FIG. 5 is a flowchart of a method for assisting the user to maintain a balanced pH level.

As discussed above, FIG. 4 is a flowchart of a method 400 for assisting the user to wear an appropriate amount of clothing in accordance with an embodiment of the present invention.

Referring to FIG. 4, in conjunction with FIGS. 1-3, in step 401, smart device 102 receives measurements of temperature and humidity from sensors 101, which are placed at various locations on a human body which are sources of sweat.

In step 402, a determination is made by smart device 102 as to whether the received measured temperature or humidity is above a first threshold (indication that user 201 will start to feel overheated) or below a second threshold (indication that user 201 will start to feel too cold). To be clear, the measured temperature and the measured humidity may each have its own separate first and second thresholds. For example, with respect to the measured temperature, the first threshold may correspond to a temperature of 90 degrees Fahrenheit and the second threshold may correspond to a temperature of 73 degrees Fahrenheit. In another example, with respect to the measured humidity, the first threshold may correspond to a relative humidity of 91% and the second threshold may correspond to a relative humidity of 65%.

If the measured temperature and humidity are not above the first threshold or below the second threshold, then smart device 102 receives subsequent measurements of temperature and humidity from sensors 101 in step 401.

If, however, the measured temperature or measured humidity is above the first threshold (indication that user 201 will start to feel overheated) or below the second threshold (indication that user 201 will start to feel too cold), then, in step 403, smart device 102 generates a notification to user 201 on smart device 102 (such as on display 315 of smart device 102) indicating to adjust the clothing layers. Adjusting the clothing layers may include adding a clothing layer, removing a clothing layer, zipping a clothing layer or unzipping a clothing layer.

For example, if the measured temperature is above a first threshold (indication that user 201 will start to feel overheated), then smart device 102 generates a notification with instructions to user 201 to remove a clothing layer (e.g., remove insulation layer 204) or unzip a clothing layer (e.g., unzip insulation layer 204). In one embodiment, the instruction as to whether to remove a clothing layer or unzip a clothing layer may depend on the degree in which the temperature is above the first threshold.

In another example, if the measured temperature is below a second threshold (indication that user 201 will start to feel too cold), then smart device 102 generates a notification with instructions to user 201 to add a clothing layer (e.g., add insulation layer 204) or zip up a clothing layer (e.g., zip up insulation layer 204). In one embodiment, the instruction as to whether to add a clothing layer or zip up a clothing layer may depend on the degree in which the temperature is below the second threshold and whether user 201 had previously been instructed to zip down a clothing layer.

In a further example, if the measured humidity is above a first threshold (indication that user 201 will start to feel overheated), then smart device 102 generates a notification with instructions to user 201 to remove a clothing layer (e.g., remove insulation layer 204) or unzip a clothing layer (e.g., unzip insulation layer 204). In one embodiment, the instruction as to whether to remove a clothing layer or unzip a clothing layer may depend on the degree in which the humidity is above the first threshold.

In another example, if the measured humidity is below a second threshold (indication that user 201 will start to feel too cold), then smart device 102 generates a notification with instructions to user 201 to add a clothing layer (e.g., add insulation layer 204) or zip up a clothing layer (e.g., zip up insulation layer 204). In one embodiment, the instruction as to whether to add a clothing layer or zip up a clothing layer may depend on the degree in which the humidity is below the second threshold and whether user 201 had previously been instructed to zip down a clothing layer.

In one embodiment, the notification includes a chime, an alert or a beep to obtain the attention of user 201. The principles of the present invention are not limited to such examples but may include any type of notification that grabs the attention of user 201.

Furthermore, in one embodiment, the notification includes detailed instructions to user 201 to add a clothing layer (e.g., add insulation layer 204), remove a clothing layer (e.g., remove insulation layer 204), zip a clothing layer (e.g., zip up insulation layer 204) or unzip a clothing layer (e.g., unzip insulation layer 204).

In this manner, the present invention assists the user in wearing an appropriate amount of clothing layers, such as informing the user to remove a clothing layer to prevent overheating or adding a clothing layer to prevent coldness, so that the user is appropriately clothed and comfortable.

In addition to assisting the user to wear an appropriate amount of clothing layers, the present invention assists the user to maintain a balanced pH level as discussed below in connection with FIG. 5.

FIG. 5 is a flowchart of a method 500 for assisting the user to maintain a balanced pH level in accordance with an embodiment of the present invention.

Referring to FIG. 5, in conjunction with FIGS. 1-3, in step 501, smart device 102 receives measurements of a pH level of the user's sweat from sensors 101, which are placed at various locations on a human body which are sources of sweat.

In step 502, a determination is made by smart device 102 as to whether the received measurement of the pH level is below a threshold (e.g., pH of 6) (indication that user 201 is approaching dehydration).

If the measured pH level is not below the threshold, then smart device 102 receives subsequent measurements of the pH level of the user's sweat from sensors 101 in step 501.

If, however, the measured pH level is below the threshold (indication that user 201 is approaching dehydration), then, in step 503, smart device 102 generates a notification to user 201 on smart device 102 (such as on display 315 of smart device 102) indicating to stop exercising and to drink water to prevent dehydration.

For example, if the measured pH level of the user's sweat is below the threshold (e.g., pH level of 6), then such an indication of acidity (a pH level that is less than 7 is acidic) may be the result of oncoming dehydration. As a result, smart device 102 generates a notification to user 201 to stop exercising and to drink water to prevent dehydration.

In one embodiment, the notification includes a chime, an alert or a beep to obtain the attention of user 201. The principles of the present invention are not limited to such examples but may include any type of notification that grabs the attention of user 201.

In this manner, the present invention assists the user in maintaining a balanced pH level thereby preventing the user from reaching a state of dehydration.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1-7. (canceled)

8. A smart device, comprising:
a memory unit for storing a computer program for assisting a user to wear an appropriate amount of clothing layers; and
a processor coupled to the memory unit, wherein the processor is configured to execute the program instructions of the computer program comprising:
receiving measurements of temperature and humidity sensed at various locations on a human body which are sources of sweat;
determining whether said measured temperature or said measured humidity is above a first threshold or is below a second threshold; and
generating a notification to said user on said smart device indicating to adjust clothing layers in response to said measured temperature or said measured humidity being above said first threshold or below said second threshold.

9. The smart device as recited in claim 8, wherein said adjusting clothing layers is selected from the group consisting of: adding a clothing layer, removing a clothing layer, zipping a clothing layer and unzipping a clothing layer.

10. The smart device as recited in claim 8, wherein said smart device is selected from the group consisting of: a smartphone, a phablet, a tablet, a smartwatch, a smart band, smart glasses and a smart key chain.

11. The smart device as recited in claim 8, wherein said temperature and humidity are measured via sensors placed within a base clothing layer.

12. The smart device as recited in claim 11, wherein said sensors are placed within said base clothing layer at the armpits of said user and along a back of said user.

13. The smart device as recited in claim 8, wherein said notification is selected from the group consisting of: a chime, an alert and a beep.

14. The smart device as recited in claim 8, wherein said notification comprises instructions to said user to add a clothing layer, remove a clothing layer, zip a clothing layer or unzip a clothing layer.

15-20. (canceled)

21. A computer program product for assisting a user to wear an appropriate amount of clothing layers, the computer program product comprising a computer readable storage medium having program code embodied therewith, the program code comprising the programming instructions for:
receiving measurements of temperature and humidity sensed at various locations on a human body which are sources of sweat;
determining whether said measured temperature or said measured humidity is above a first threshold or is below a second threshold; and
generating a notification to said user on a smart device indicating to adjust clothing layers in response to said measured temperature or said measured humidity being above said first threshold or below said second threshold.

22. The computer program product as recited in claim 21, wherein said adjusting clothing layers is selected from the group consisting of: adding a clothing layer, removing a clothing layer, zipping a clothing layer and unzipping a clothing layer.

23. The computer program product as recited in claim 21, wherein said smart device is selected from the group consisting of: a smartphone, a phablet, a tablet, a smartwatch, a smart band, smart glasses and a smart key chain.

24. The computer program product as recited in claim 21, wherein said temperature and humidity are measured via sensors placed within a base clothing layer.

25. The computer program product as recited in claim 24, wherein said sensors are placed within said base clothing layer at the armpits of said user and along a back of said user.

26. The computer program product as recited in claim 21, wherein said notification is selected from the group consisting of: a chime, an alert and a beep.

27. The computer program product as recited in claim 21, wherein said notification comprises instructions to said user to add a clothing layer, remove a clothing layer, zip a clothing layer or unzip a clothing layer.
